(19) United States
(12) Patent Application Publication
(10) Pub. No.: US 2014/0142000 A1
(43) Pub. Date: May 22, 2014

(54) MICROFLUIDIC ARRAY PLATFORM FOR SIMULTANEOUS CELL CULTURE UNDER OXYGEN TENSIONS

(71) Applicant: Academia Sinica, Taipei (TW)

(72) Inventors: Yi-Chung Tung, Taipei (TW); Chien-Chung Peng, Taipei (TW); Hsiu-Chen Shih, Taipei (TW); Wei-Hao Liao, Taipei (TW)

(73) Assignee: ACADEMIA SINICA, Taipei (TW)

(21) Appl. No.: 14/059,297

(22) Filed: Oct. 21, 2013

Related U.S. Application Data
(60) Provisional application No. 61/718,478, filed on Oct. 25, 2012.

(51) Int. Cl. G01N 33/50

(52) U.S. Cl. CPC G01N 33/5008
USPC 506/10; 506/40

(57) ABSTRACT

The present invention relates to a microfluidic array platform comprising a substrate and two layers between which one membrane is sandwiched, wherein a plurality of cell culture wells are constructed in the top layer and one or more microfluidic channels for oxygen scavenging reactions or/and oxygen generating reactions to control the oxygen tensions are constructed in the bottom player. The microfluidic array platform is capable of simultaneously performing cell culture under different oxygen tensions and compatible with existing cell incubators and high-throughput instruments for cost-effective setup and straightforward operation.

### CROSS REFERENCE

This application claims priority to U.S. Provisional Patent Appl. No. 61/718,478, filed on Oct. 25, 2012, the contents of which is incorporated by reference in its entirety.

### FIELD OF THE INVENTION

The present invention relates to a microfluidic array platform for simultaneous cell culture under oxygen tensions.

### BACKGROUND OF THE INVENTION

Oxygen plays an essential role in biological systems, and modulates cellular functions in vivo. To date, in vitro cell culture studies have been primarily accomplished under atmospheric conditions of approximately 20% oxygen (O₂). However, in a human body, cells respond to a wide range of oxygen tensions. (Decaris et al., Angiogenesis, 12:303, 2009.) For instance, normal brain oxygen levels range from 5% to 10%, and oxygen level in alveolar is about 14%. (McCord et al., Mol. Cancer. Res., 7:489, 2009.) Oxygen affects cellular response in various ways, including metabolic pathways and plasma membrane integrity. Therefore, oxygen tension plays an important role in regulating various cellular functions in both normal physiology and disease states. (Allen & Bhatia, Biotechnol. Bioeng., 82:253-262, 2003.) For example, hypoxiainducible genes regulate several biological processes, including cell proliferation, angiogenesis, metabolism, apoptosis, immortalization, and migration. Hypoxia-inducible factor-1 (HIF-1) is required in normal embryogenesis for normal vascular development at an early stage. (Harris, Nat. Rev. Cancer, 2:38, 2002.) Angiogenesis has been recognized as an essential element in tumor growth. The expression of the potent angiogenesis stimulator, vascular endothelial growth factor (VEGF), is upregulated by hypoxia. Given the above, oxygen gradient is a key factor in tumor growth and progression. (Rice & Huang, Cancer Manage. Res., 3: 9, 2011; Giordano & Johnson, Curr. Opin. Genet. Dev., 11:35, 2001.)

Conventionally, the oxygen tensions for cell culture were controlled by direct bubbling of oxygen or nitrogen gas into the culture medium to create environments with various oxygen tensions. (Allen & Bhatia, Biotechnol. Bioeng., 82:253-262, 2003.) However, the methods often require complicated instrumentation and a large volume of gas supply. Then, a direct addition of an oxygen scavenging agent into cell culture media was utilized for oxygen tension control due to its simplicity and efficiency. But, the chemical addition would alter the medium compositions, and further affect cellular responses. (Reist et al., J. Neurochem., 71: 2431, 1998.) The aforementioned conventional methods cannot achieve oxygen gradient generation with high spatial resolution. To generate desired oxygen gradients for cell culture, a number of microfluidic devices have been developed. For example, an elastomer bioreactor capable of generating axial oxygen gradient caused by the uptake of oxygen by cells inside microfluidic channels was developed. (Mehta et al., Biomed. Microdevices, 9:123, 2007.) Some polymers with low oxygen diffusivities were exploited to construct hard top soft bottom microfluidic devices to further deplete the oxygen inside the microfluidic channels. (Mehta et al., Anal. Chem., 81:3714, 2009.) Similarly, impermeable capillaries were employed to generate mass-transfer gradients, including oxygen, inside the elastomer microfluidic device. (Pinelis et al., Biomed. Microdevices, 10:807, 2008.) To dynamically control oxygen profiles, an oxygen microgradient array (OMA) device using water electrolysis controlled by electrode patterns was developed. (Park et al., Lab Chip, 6:611, 2006.) In addition, a multi-layer microfluidic device was constructed using a gas-permeable membrane and a computer-controlled multi-channel gas mixer. The device can generate oxygen gradients with arbitrary shapes in a microfluidic device. (Adler et al., Lab Chip, 10:388, 2010.) Recently, it was first reported that oxygen scavenging liquids with a gas permeable membrane were used to control the oxygen gradients without altering cell culture medium compositions. (Skolimowski et al., Lab Chip, 10:2162, 2010.) However, the existing microfluidic cell culture devices with oxygen gradients face several challenges that hinder their practical usage in biological labs. For example, to use oxygen and nitrogen gases for oxygen gradient generation requires precise flow control instruments, tedious interconnections, and bulky gas cylinders to store compressed gases. Because the gas can easily penetrate through the permeable membrane that may cause medium evaporation and bubble generation inside the cell culture channel, the entire setup is unreliable for long-term studies, and cannot be directly implemented into the conventional cell incubators.

A microfluidic device with a single-layer pattern to generate oxygen gradients across a microfluidic channel for cell culture was developed. (Chen et al., Lab Chip, 11: 3626-3633, 2011) The single-layer construction makes cellular microscopic observation straightforward and device fabrication easier. By confining the areas for chemical reactions, the device can control the oxygen tensions efficiently using minimal chemicals without altering the surrounding gaseous compositions. The device takes advantage of the spatially confined chemical reaction method to simultaneously generate multiple oxygen tensions for cell culture. The localized chemical reactions eliminates the crosstalk between cell culture sites, and provides the device having great compatibility of a cell incubator.

However, it is still desirable to develop a device for simultaneous performing cell culture under various oxygen tensions.

### BRIEF SUMMARY OF THE INVENTION

In the present invention, a new microfluidic array platform is provided, which is capable of simultaneously performing cell culture under various oxygen tensions.

Accordingly, in one aspect, the invention provides a microfluidic array platform for simultaneous cell culture under oxygen tensions. The microfluidic array platform comprises:

a substrate;
a membrane;
two layers on the substrate, including a top layer for cell culture on the membrane, and a bottom layer for oxygen tension control underneath the membrane; wherein the membrane is sandwiched between the top layer and the bottom layer;
a plurality of cell culture wells constructed in the top layer;
one or more microfluidic channels constructed in the bottom layer, which are exploited for oxygen scavenging reactions or/and oxygen generating reactions to control the oxygen tensions in the cell culture wells, wherein each of the microfluidic channels has two or more separate inlets for introducing chemicals for oxygen scavenging reactions or/and oxygen generating reactions.

In the other aspect, the present invention provides a method for simultaneous cell culture under oxygen tensions using the microfluidic array platform according to the invention.

### BRIEF DESCRIPTION OF THE DRAWINGS

For the purpose of illustrating the invention, there are shown in the drawings embodiments which are presently preferred. It should be understood, however, that the invention is not limited to the preferred embodiments shown.

In the drawings:

FIG. 1(a) provides an image showing the schematic of the platform with PDMS layers of the microfluidic cell culture array according to the invention.

FIG. 1(b) provides an image showing the fabricated platform filled with food dyes, including the chemical reactants mixing areas and the gas exchange areas; wherein the gas exchange areas with different food dye colors represents cell culture wells with different oxygen tensions.

FIG. 2(a) shows the experimental setup for oxygen tension characterization based on the relative fluorescence lifetime measurement of an oxygen sensitive dye.

FIG. 2(b) shows the experimentally estimated oxygen tensions generated inside a cell culture well at the surface on which cells are cultured using 1M NaOH and pyrogallol at flow rates of 20 μl min⁻¹ at various concentrations.

FIG. 3(a) provides an image showing the fabricated microfluidic array platform after cell culture with drug treatments and PrestoBlue cell viability assay under various oxygen tensions on a single chip.

FIG. 3(b) shows the relative A549 cell viabilities (normalized to the cell cultured in the growth medium under normoxia for 12 hours) after 12 hours hypoxia-activated anti-cancer drug (TPZ) treatments under various oxygen tensions using the array platform according to the invention, wherein the data were expressed as the mean±SD.

FIG. 4 shows a comparison of cell viabilities under various drug treatments in normoxia and hypoxia using the cell culture arrays according to the invention and conventional 96-well plate, wherein the data are expressed as the mean±s.e.m. (n=4 for the device experiments; n=8 for the well plate experiments).

FIG. 5 shows the results of the normalized cell viability (PrestoBlue) experiments of the cells under various drug treatment and oxy using the device according to the invention (control and drug treatments under multiple oxygen tensions), which shows Presto color variation after reacting with cell culture medium with different living cell populations.

### DETAILED DESCRIPTION OF THE INVENTION

Unless otherwise defined, all technical and scientific terms used herein have the same meaning as commonly understood by one of ordinary skill in the art to which this invention pertains.

As used herein, the singular forms “a”, “an”, and “the” include plural referents unless the context clearly dictates otherwise. Thus, for example, reference to “a sample” includes a plurality of such samples and equivalents thereof known to those skilled in the art.

The invention provides a microfluidic array platform for simultaneous cell culture under oxygen tensions. The microfluidic array platform comprises:

a substrate;
a membrane;
two layers on the substrate, including a top layer for cell culture on the membrane, and a bottom layer for oxygen tension control underneath the membrane; wherein the membrane is sandwiched between the top layer and the bottom layer;
a plurality of cell culture wells constructed in the top layer;
one or more microfluidic channels constructed in the bottom layer, which are exploited for oxygen scavenging reactions or/and oxygen generating reactions to control the oxygen tensions in the cell culture wells, wherein each of the microfluidic channels has two or more separate inlets for introducing chemicals for oxygen scavenging reactions or/and oxygen generating reactions.

According to the present invention, the microfluidic array platform is capable of simultaneously performing cell culture under various oxygen tensions. The microfluidic array platform takes advantage of microfluidic phenomena while exhibiting the combinatorial diversities achieved by microarrays. Importantly, the microfluidic array platform is compatible with existing cell incubators and high-throughput instruments for cost-effective setup and straightforward operation.

In one embodiment of the invention, the substrate is made from glass. The membrane or layers may be made from an elastomeric material that has excellent optical transparency, manufacturability and high gas permeability. A preferable example of the elastomeric material is polydimethylsiloxane (PDMS), which has been broadly exploited to construct microfluidic platforms, including cell culture wells, and microfluidic channels for oxygen scavenging reactions or/and oxygen generating reactions to control oxygen tensions.

According to the invention, the microfluidic array platform is designed to have two layers, including one top layer for cell culture and one bottom layer for oxygen tension control. The two layers are separated by a membrane. That is, the membrane is sandwiched between the two layers. The thickness of the membrane may be from 20 μm to 1000 μm, preferably 50 μm to 500 μm. In one preferable example of the microfluidic array platform, the two layers are separated by a membrane with a thickness of about 200 μm. The array platform according to the invention may be in any form that is appropriate and convenient for manufacturing and using the platforms. In one embodiment of the invention, the array is on a chip.

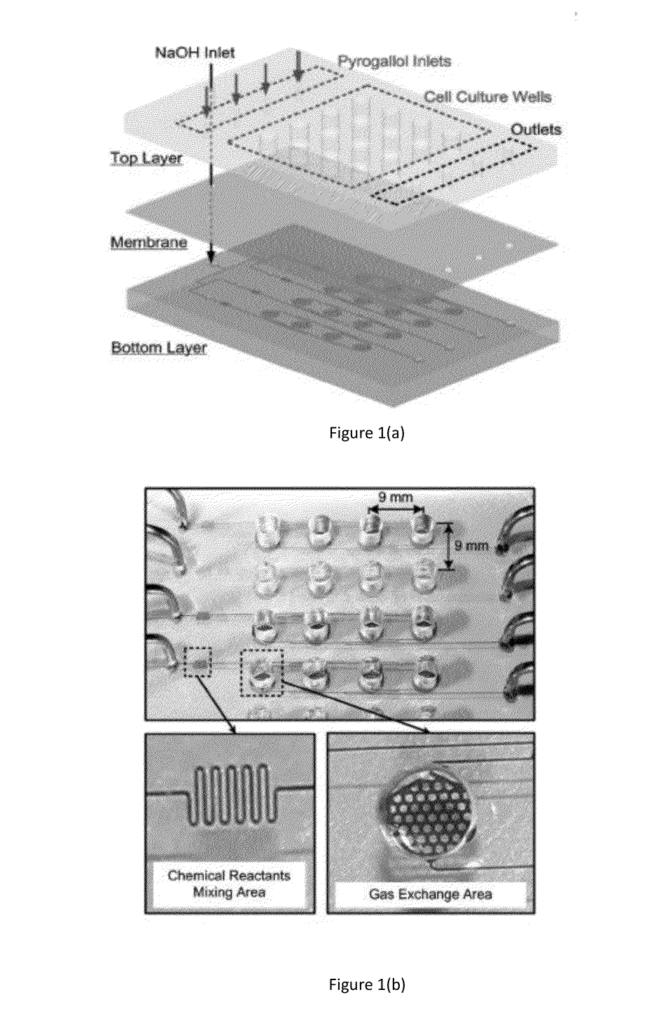

Referring to FIGS. 1A, 1B and 1C as an embodiment of the microfluidic cell culture array platform, the top layer may be designed to be one or more micro-containers, such as wells, in any form for cell culture. As shown in FIG. 1C, a plurality of cell culture wells are constructed in the top layer, which are arranged in a matrix form, such as the same dimension as a standard 96-well plate (9 mm apart in both directions) commonly used in the biological laboratory. Each of the cell culture wells may be in a size of about 4 mm in diameter.

Referring to FIG. 1B as an embodiment of the microfluidic array platform, the microfluidic channels constructed in the cell culture wells. The oxygen tensions are controlled by oxygen scavenging reactions or/and oxygen generating reactions. The oxygen scavenging reaction may be a spatially confined chemical reaction method that previously developed and disclosed by the inventors, (Chen et al., Lab Chip, 11:3626-3636, 2011), the entire contents of which are hereby incorporated by reference. On the other hand, the oxygen tension can also be controlled by oxygen generations. In order to efficiently scavenge oxygen, any chemical that can scavenge oxygen, such as pyrogallol (benzene-1,2,3-triol, C₆H₆O₃) and sodium hydroxide (NaOH), may be used. On the other hand, in order to efficiently generate oxygen, any chemical that can generate oxygen, or a chemical that can decompose hydrogen peroxide (H₂O₂) such as sodium sulfite with cobalt salt, can be used. The chemicals may be introduced into the microfluidic array from two or more separate inlets. The chemicals start to mix and react with each other when flowing through the meander-shape channels. The widths of the microfluidic channels underneath the cell culture wells are increased to enlarge the gas exchange areas for efficient oxygen tension control. The membrane sandwiched between the layers prevents the cell culture medium directed contacting the chemicals, while maintains excellent oxygen permeability.

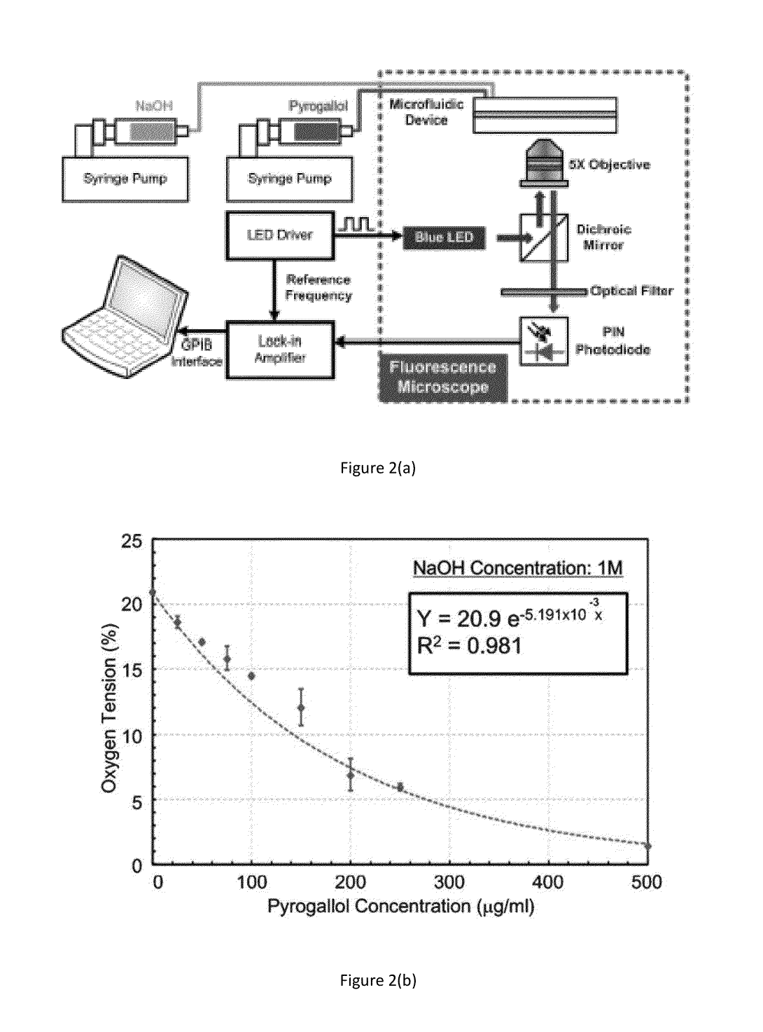

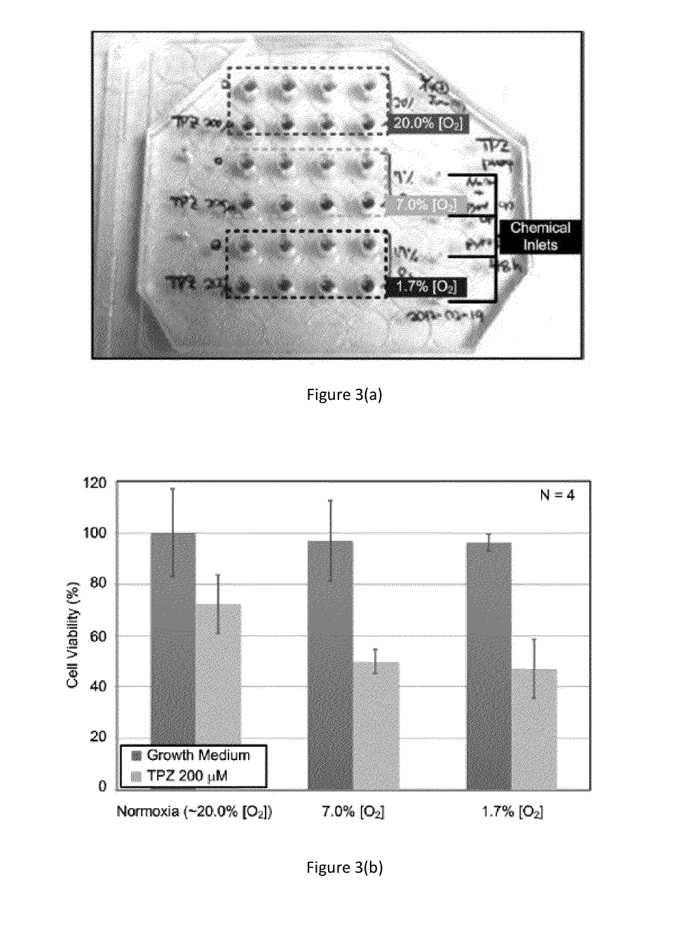

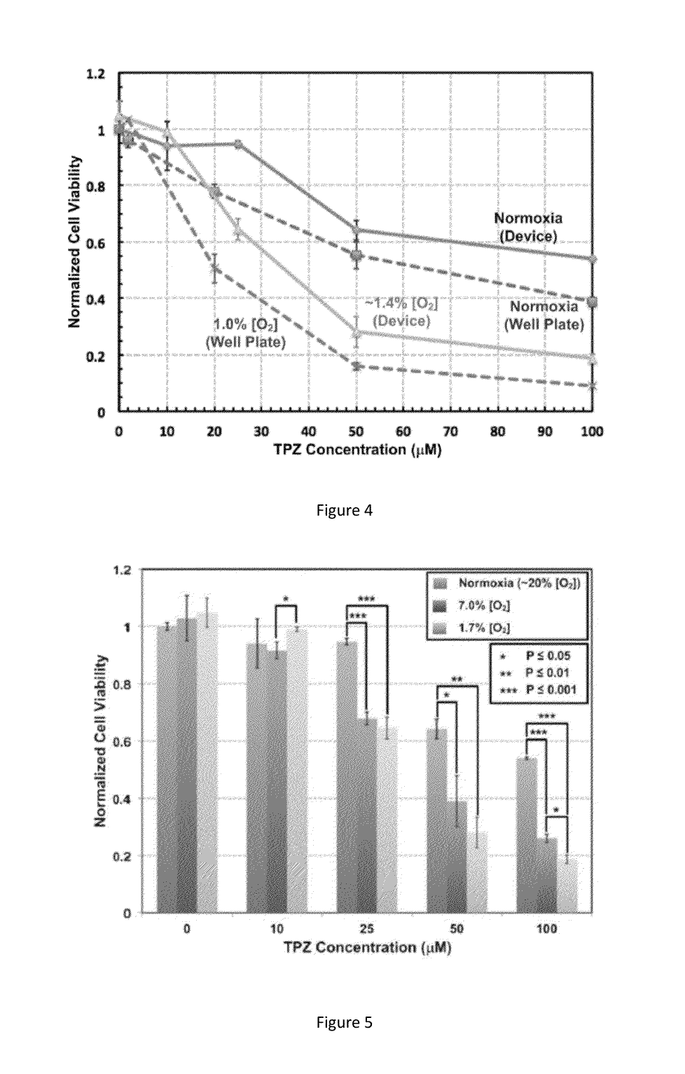

In one embodiment of the claimed array platform according to the invention, the oxygen tensions in the cell culture wells are calibrated by measuring the relative fluorescence lifetime of an oxygen sensitive fluorescence dye, tris(2,2′-bypyridyl) ruthenium (III) chloride, while flowing chemicals in the underneath channels with various concentrations using the experimental setup as shown in FIG. 2. The calibration results were shown in FIGS. 3 and 5, which demonstrate the oxygen tension controllability of the array platform. The platform can achieve oxygen tensions from less than 2% to normoxia in a single platform without interfering gas contents in a cell incubator. In one experiment for testing the performance of the array platform according to the invention, a test using a hypoxia-activated anti-cancer drug, triapazamine (TPZ), on human alveolar epithelia cell (A549) was performed and the experimental results were shown in FIG. 4B, which indicated the cell viabilities of the drug treated A549 cells decreased more than 20% under low oxygen tensions compared to normoxia.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated herein by reference in their entirety.

### EXAMPLE

### Example 1

### Microfluidic Cell Culture Array Design and Fabrication

The microfluidic cell culture array comprises a glass substrate and two polydimethylsiloxane (PDMS) layers: a top layer for cell culture and a bottom layer for chemical reactions for oxygen tension control. The two layers are separated by a PDMS membrane with a thickness of 200 μm as shown in FIG. 1(a). On the top layer, the cell culture wells are arranged in the same dimensions as a standard 96-well plate commonly used in biological labs. The top layer contains 4 rows and 4 columns of 4 mm diameter cell culture wells and they are 9 microfluidic channels constructed on the bottom layers are exploited for oxygen scavenging chemical reactions to control oxygen tensions in the cell culture wells. In order to efficiently scavenge the oxygen, the chemical reactants are introduced into the platform from two separated inlets, and start to mix and react with each other when flowing through the meander-shape channels as shown in FIG. 1(b). The widths of the microfluidic channels underneath the cell culture wells are increased to enlarge the gas exchange areas for efficient oxygen tension control. Furthermore, the microfluidic channels at the nottom layer underneath the cell culture wells are designed with honeycomb shapes to provide mechanical support of the membranes, on which the cells are cultureed. The membrane sandwiched between the PDMS layers prevents the cell culture medium directly contacting the chemical reactants, while maintains excellent oxygen permeability (diffusion coefficient of oxygen in pure PDMS at 25° C., D=3.55×10⁻⁵ cm² s⁻¹).

The microfluidic cell culture array platform is fabricated using the well-developed multi-layer soft lithography (MSL) technique. The top layer with cell culture wells is fabricated using a 4 mm-diameter biopsy punch on a blank PDMS slab. The PDMS precursor with 1:10 (v/v) curing agent to base ratio is cured at 60 μC for more than 4 hours to fabricate the top layer. The bottom layer with the microfluidic channel patterns is fabricated by the soft lithography replica molding process. The mold is fabricated by patterning a negative tone photoresist (SU-8 2050, MicroChem. Co., Newton, Mass.) on a silicon wafer using a conventional photolithography process. The mold is then silanized with 1H,1H,2H,2H-perfluorooctyltrichlorosilane (78560-45-9, Alfa Aesar, Ward Hill, Mass.) in a desiccator for more than 30 min. at room temperature to prevent undesired bonding of PDMS to the mold. The aforementioned PDMS precursor is then poured on the mold and cured for more then 4 hours. In addition, the PDMS membrane with thickness of 200 μm is prepared by spinning the aforementioned PDMS precursor onto a silanized silicon wafer. After fabricating each layer, the top layer is first bonded with the PDMS membrane using oxygen plasma surface treatment (90 W for 45 s), and interconnection inlets and outlets are punched on the bonded layer. The bonded layer is then aligned and bonded with the bottom layer using the same oxygen plasma surface treatment.

The bottom layer was prepared by spinning the aforementioned PDMS precursor on top of a microscope glass slide or an indium tin oxide (ITO)-coated glass slide treated with oxygen plasma (PX-250, Nordson MARCH Co., Concord, Calif.) at 90 W for 40 s. The PDMS coated glass slide was then cured on a 150° C. hotplate for more than 20 minutes. Afterwards, the PDMS channel layer and the bottom glass slide were surface-treated using the oxygen plasma at 90 W for 40 s and sealed against each other. The bonded chip was further cured on the 150° C. hotplate for additional 20 minutes to promote the bonding and assure full curing of the PDMS to enhance cell compatibility.

### Example 2

### Numerical Simulation

Using the device according to the invention, oxygen tension uniformity through the entire cell culture well and its transient response were simultaneously measured to obtain a numerical simulation to study the device performance. A finite element analysis (FEA) model is constructed using COMSOL Multiphysics (Ver. 4.3, COMSOL Inc., Burlington, Mass.) commercial software. To simplify the simulation, one set of the honeycomb shaped microfluidic channel for oxygen scavenging was modeled in the simulation. The geometries of the model were set to be identical to the designed device. A flow with depleted oxygen was introduced into the channel with a flow rate identical to the experimental one. The material properties of PDMS and water were applied to the channel structure and flow, respectively. The transient oxygen tension profiles right after the flow introduction were simulated. The oxygen tension profiles at different time points after the flow introduction were obtained. As shown in the simulation results, the entire gas exchange area, including fluidic channels and PDMS honeycomb structures, reached uniform oxygen tension distribution approximately 100 s after the flow introduction. The oxygen tension differences were less than 0.5% through the entire gas exchange area. The results suggested that the device design be capable of generating uniform oxygen tension inside the cell culture wells. Furthermore, the oxygen tension could reach a steady state within a short period (approximately 5 min.) in the simulation. Therefore, the chemical reactants could be refilled for long-term experiments with minimum disturbance of oxygen tensions, which was desired for most cell culture applications.

### Example 3

### Cell Culture and Anti-Cancer Drug Test Using the Device According to the Invention

To examine the performance of the array on a chip form for cell culture, carcinomic human alveolar basal epithelial cells (A549, ATCC, Manassas, Va., USA) were utilized. A549 cells were cultured in F-12K medium (Gibco 21127, Invitrogen Co., Carlsbad, Calif., USA) with 10% v/v fetal bovine serum (FBS) (Gibco 10082, Invitrogen Carlsbad, Calif., USA) and 1% v/v antibiotic-antimycotic (Gibco 15240, Invitrogen, Carlsbad, Calif., USA). The stocks were maintained under 5% CO₂ in T25 cell culture flasks (Nunc 156367, Thermo Scientific Inc., Rochester, N.Y., USA), and passaged by dissociation with 0.25% trypsin-EDTA (Gibco 25200, Invitrogen, Carlsbad, Calif., USA). Cell suspensions for the experiments were made by centrifugation of dissociated cells at 1000 rpm for 3 min. at room temperature. The microfluidic channel was treated with the extracellular matrix (ECM) protein, fibronectin (F2006, Sigma-Aldrich Co., St Louis, Mo., USA), at a concentration of 100 mg per ml for more than 2 hours inside the incubator before introducing the cell suspension into the chip. The cell suspension with a density of 2000 cells in 60 μl growth medium was then introduced into the cell culture wells of the array platform on a chip according to the invention, and incubated overnight to promote cell adhesion onto the cell culture wells of the chip.

For oxygen scavenging, pyrogallol (benzene-1,2,3-triol, C₆H₆O₃) (87-66-1, Alfa Aesar, Ward Hill, Mass., USA) and sodium hydroxide (NaOH) (30620, Sigma-Aldrich, St Louis, Mo., USA) were introduced into the microfluidic channels to control oxygen tensions. The oxygen tensions in the cell culture wells were calibrated by measuring the relative fluorescence lifetime of an oxygen sensitive fluorescence dye, tris(2,2′-bypyridyl) ruthenium (III) chloride (50525-27-4, Acros Organics, Geel, Belgium), while flowing chemicals in the underneath channels with various concentrations using the experimental setup as shown in FIG. 2(a). The setup is based on an inverted fluorescence microscope with a LED and a PIB photodiode as a light source and a photo detector, respectively. The oxygen tensions in the cell culture wells of the microfluidic array platform according to the invention were measured, while introducing chemical reactants with various concentrations (NaOH at the concentration of 1M, and pyrogallol at the concentration of from 0 to 500 μg/ml) for oxygen scavenging into the microfluidic channels at the bottom layer. The calibration results was shown in FIG. 2(b). It was indicated that the platform achieved oxygen tensions from less than 2% to normoxia in a single platform without interfering gas contents in a cell incubator.

To demonstrate the capability of generating oxygen gradients for cell studies using the array platform on a chip according to the invention, the tests for cell culture with continuous anti-cancer drug medium perfusion under oxygen gradients were conducted. In this experiment, an anti-cancer drug, Triapazamine (TPZ, 3-amino-1,2,4-benzotriazine-1,4-dioxide) (Toronto Research Chemicals Inc., North York, Canada), which is activated to a toxic radical only at very low levels of oxygen, was utilized, referring to the methods as described by Brown (Brown, Cancer Res., 59: 5863, 1999; Marcu and Olver, Curr. Clin. Pharmacol., 1: 71, 2006), the experiment contents of which are herein incorporated by reference.

The relative cell viabilities (normalized to the cell cultured in the growth medium under normoxia for 12 hours) of the drug treated A549 cells were measured. The results as shown in FIGS. 3(a) and 3(b) indicated that the cell viabilities of the drug treated cells decreased more than 20% under low oxygen tensions compared to normoxia.

### Example 4

### Comparison of Cell Viabilities Under Various Drug Treatments in Normoxia and Hypoxia

A comparison of cell viabilities under various drug treatments in normoxia and hypoxia conditions using the cell culture array platform according to the invention and conventional 96-well plates (167008, Nunclon DELTA Surface, Nunc) with an oxygen tension-controlled cell incubator (Heracell 240i, Thermo Scientific). In the well plate experiments, approximate 5000 A 549 cells were seeded into each well. The cells with 80 μl medium were incubated overnight in the cell incubator to ensure the cell attachment onto the wells before the drug treatments, and 20 μl drug solutions with various TPZ concentrations were than added into the wells. The cells were incubated with drug-contained medium for 48 hours. After the testing, the cell viabilities were estimated using the fluorescence-based cell viability assay, and read by the plate reader. As shown in FIG. 4, the both's performances were similar for both normoxia and hypoxia conditions. The slightly low viabilities in the well plate experiences might result from the low gas oxygen permeability of polystyrene used to make well plates. Therefore, the real oxygen tensions that cells experienced would be lower than that set in the incubator. As a result, the drug showed more hypoxia-activated cytotoxicity of TPZ to A549 cells comparing to that obtained using the device according to the invention. The cell morphology differences suggest that the oxygen tensions have been successfully controlled using the device according to the invention, and the results represent the hypoxia-activated cytotoxicity of TPZ.

### Example 5

### Cell Culture and Anti-Cancer Drug Test Using the Device According to the Invention

In the experiments, 1 M NaOH and pyrogallol solutions with concentrations of 0, 190, and 500 μg ml⁻¹ at flow rates of 20 μml⁻¹ were used to set the oxygen tensions inside the cell culture wells as normoxia (˜20%), 7.0%, and 1.4%, respectively, and the drug, TPZ, with concentrations of 0, 10, 25, 50, and 100 mM were applied. The cells were cultured under different conditions, including: 20% oxygen and 0 mM TPZ; 20% oxygen and 50 mM TPZ; and 1.4% oxygen and 50 mM TPZ, for 48 hours. It was found that the cells reached confluency and attached well onto the PDMS membrane substrate in the culture well under the control conditions (−20% oxygen and 0 mM TPZ). Furthermore, the cell viability was checked using a live/dead stain solution containing calcein AM (1 mM) and ethidium homodimer-1 (2 mM) from LIVE/DEAD Viability/Cytotoxicity Kit (L3224, Invitrogen). The great viability (>95% by analyzing four fluorescence images taken in different cell culture wells using an image analysis software MetaMorph from Molecular Devices, Sunnyvale, Calif.) of the cells cultured under the control conditions for 48 hours confirmed the great cell compatibility of the multi-layer PDMS microfluidic device according to the invention. In contrast, the results of the cells cultured under 20% oxygen and 50 mM TPZ showed that the shapes of the cells became rounded and some cells detached from the substrate due to the cytotoxicity of TPZ. Furthermore, the result of the cells cultured under 1.4% oxygen and 50 mM TPZ showed that more cells became rounded and detached from the substrate. To quantitatively characterize the cell viabilities of A549 cells treated with TPZ under various oxygen tensions, the PrestoBlue cell viability assays were performed in the experiments. The developed microfluidic array platform can be directed placed into a microplate reader commonly used in biological labs for assay readout. The measurement results of the cells cultured under different oxygen tension were shown in FIG. 5. In order to minimize variations between the experiments, the measured fluorescence intensity from each well was normalized to the average intensity of the cell culture wells, in which the cells were cultured under normoixa without TPZ (i.e. 0 mM TPZ). To compare the differences between the normalized cell viabilities for various oxygen tensions, statistical analysis, unpaired, two-tailed Student's t-tests were performed in this study. As can be seen in the figure, the cells cultured under various oxygen tensions (−20% to 1.4%) without TPZ treatments have similar normalized cell viabilities. The results suggest that the method exploited in the developed device to control oxygen tensions is not cytotoxic. Therefore, the developed array platform possesses great cell compatibility and can be utilized for various cell studies. Furthermore, for the TPZ treatments with concentrations higher than 25 mM, the normalized cell viabilities for the normoxia culture conditions are more than 20% higher than those for the culture under low oxygen tensions (7.0% and 1.4%). In addition, the statistical analysis also shows a significant difference between the normoxia and low oxygen tension conditions. When the concentration of TPZ was increased to 100 mM, the normalized cell viability for the 1.4% oxygen tension condition was even lower statistically than that for the 7.0% oxygen tension condition. The drug testing results were further compared to those accomplished using conventional well plates and an oxygen-tension controlled cell incubator setup.

Given the above, the comparison demonstrates that the results obtained from the method according to the invention and the standard 96-well plates are similar. Consequently, the experimental results demonstrate that oxygen tensions can be straightforwardly controlled using the developed microfluidic cell culture array, and show the hypoxia-activated cytotoxicity of TPZ. Moreover, the experimental results also show that oxygen tension play an essential role to not only regulate cell behaviors but also affect the effectiveness of drugs according to their modes of actions.

I/We claim:

1. A microfluidic array platform for simultaneous cell culture under oxygen tensions, which comprises:
a substrate;
a membrane;
two layers on the substrate, including a top layer for cell culture on the membrane, and a bottom layer for oxygen tension control underneath the membrane; wherein the membrane is sandwiched between the top layer and the bottom layer;
a plurality of cell culture wells constructed in the top layer;
one or more microfluidic channels constructed in the bottom layer, which are exploited for oxygen scavenging reactions or/and oxygen generating reactions to control the oxygen tensions in the cell culture wells, wherein each of the microfluidic channels has two or more separate inlets for introducing chemicals for oxygen scavenging reactions or/and oxygen generating reactions.

2. The microfluidic array platform of claim 1, wherein the substrate is made from glass.

3. The microfluidic array platform of claim 1, wherein the layers and membrane are made from an elastomeric material having optical transparency, manufacturability and high gas permeability.

4. The microfluidic array platform of claim 3, wherein the layers and membrane are made from polydimethylsiloxane (PDMS).

5. The microfluidic array platform of claim 1, wherein the membrane sandwiched between the two layers is one with a thickness of 20 μm to 1000 μm.

6. The microfluidic array platform of claim 5, wherein the membrane sandwiched between the two layers is one with a thickness of 50 μm to 500 μm.

7. The microfluidic array platform of claim 6, wherein the membrane sandwiched between the two layers is one with a thickness of 200 μm.

8. The microfluidic array platform of claim 1, which is in the form of a chip.

9. The microfluidic array platform of claim 1, wherein the cell culture wells are arranged in a matrix form.

10. The microfluidic array platform of claim 1, wherein the oxygen tensions in the cell culture wells are controlled by a spatially confined chemical reaction method.

11. The microfluidic array platform of claim 10, wherein the chemicals for oxygen scavenging or/and generating reactions are introduced into the platform from separate inlets.

12. The microfluidic array platform of claim 11, wherein the chemicals for oxygen scavenging reactions are pyrogallol and NaOH.

13. The microfluidic array platform of claim 11, wherein the chemical for oxygen generating reactions is a chemical that can decompose hydrogen peroxide.

14. The microfluidic array platform of claim 11, wherein the chemical for oxygen generating reactions is sodium sulfite with cobalt salt.

15. The microfluidic array platform of claim 1, wherein the microfluidic channels are meander-shaped.

16. The microfluidic array platform of claim 1, wherein the chemicals start to mix and react with each other when flowing through the channels.

17. A method for simultaneous cell culture under oxygen tensions using the microfluidic array platform according to claim 1.
